SEQUENCE LISTING (4 sequences)

SEQ ID NO: 1 - DNA, 11 bp, Artificial sequence
  Description of Artificial Sequence Synthetic oligonucleotide
tcgactgcgtg

SEQ ID NO: 2 - DNA, 29 bp, Artificial sequence
  Description of Artificial Sequence Synthetic oligonucleotide
cgtatgccgtcttctgcttgcacgcagtc

SEQ ID NO: 3 - DNA, 65 bp, Artificial sequence
  Description of Artificial Sequence Synthetic oligonucleotide
  modified_base (1)..(1): phosphorylation modification
  misc_feature (25)..(31): n is a, c, g or t
  modified_base (63)..(64): thio modification
caagcagaagacggcatacgagatnnnnnnngtgactggagttcagacgtgtgctcttccgatct

SEQ ID NO: 4 - DNA, 57 bp, Artificial sequence
  Description of Artificial Sequence Synthetic oligonucleotide
  modified_base (1)..(1): phosphorylation modification
gatcggaagagcgtcgtgtagggaaagagtgtagatctcggtggtcgccgtatcatt

(19) United States
(12) Patent Application Publication
(10) Pub. No.: US 2022/0348911 A1
(43) Pub. Date: Nov. 3, 2022

(54) METHOD TO CONSTRUCT WHOLE-GENOME HIGH-THROUGHPUT SEQUENCING LIBRARY AND TEST KIT THEREOF

(71) Applicant: BERRY GENOMICS CO., LTD., Beijing (CN)

(72) Inventors: Jianguang Zhang, Beijing (CN); Di Chen, Beijing (CN); Ling Shang, Beijing (CN)

(21) Appl. No.: 17/674,128

(22) Filed: Feb. 17, 2022

(30) Foreign Application Priority Data
Dec. 29, 2020 (CN) 202011584655.X

(51) Int. Cl. C12N 15/11; C40B 50/06; C12Q 1/6806; C12Q 1/6869; C12N 15/10

(52) U.S. Cl. CPC C12N 15/11; C40B 50/06; C12Q 1/6806; C12Q 1/6869; C12N 15/1093

(57) ABSTRACT

The present disclosure relates to a method for constructing a whole genome high-throughput sequencing library comprising the following steps: (1) extracting a sample gDNA; (2) fragmenting said sample gDNA by enzyme cleavage, filling ends of the gDNA and adding A base to the gDNA fragments to obtain an A-added gDNA; (3) connecting the A-added gDNA with a linker combination to obtain a connected produce, said linker combination comprises two parts: a Y-shaped reverse linker and a high GC clamp linker; (4) purifying said connected product to obtain a purified product; and (5) screening the fragment of said purified product to obtain a sequencing library. The present disclosure also relates to a kit for constructing a whole genome high-throughput sequencing library.

### CROSS-REFERENCE TO RELATED APPLICATIONS

This application claims the benefit of priority to Chinese Patent Application No. 202011584655.X, filed on Dec. 29, 2020. The entire contents of the aforementioned application are incorporated herein by reference.

### TECHNICAL FIELD

The present disclosure relates to a method and a kit for constructing a whole genome high-throughput sequencing library. More specifically, the disclosure relates to a method and a kit for constructing a whole genome high-throughput sequencing library that can reduce redundancy and sequence index hopping.

### SEQUENCE LISTING

The instant application contains a Sequence Listing which has been filed electronically in ASCII format and is hereby incorporated by reference in its entirety. Said ASCII copy, created on Mar. 9, 2022 and having a size of 1,668 bytes, is named 136186_00402_SL.txt.

### BACKGROUND

Whole Genome Sequencing (WGS) is a method for sequencing the whole genome of different human individuals or populations by using high-throughput sequencing platforms and performing bioinformatics analysis at the individual or population level. It can comprehensively explore genetic variants at the DNA level, providing important information for screening disease causative and susceptible genes and studying pathogenesis and genetic mechanisms. Compared with whole-exome sequencing, whole genome sequencing has its unique advantages due to the results contain complete and rich information, which can obtain more information than that obtained by exome sequencing or targeted sequencing. In recent years, whole genome sequencing has become accessible due to the continuous advancement of sequencing technology and reduction of sequencing cost. Moreover, whole genome sequencing is more advantageous in identifying single nucleotide variants (SNPs), insertion and deletion mutations (Indel), so whole genome sequencing is gradually becoming another option for clinical and basic research.

Regarding the method for preparing whole genome sequencing libraries, it can be classified as two library preparation methods: those with PCR amplification and those without PCR amplification (PCR-free). Comparing the two library preparation methods, the advantage of the PCR process is that it requires less DNA template volume, and the disadvantage is that the operation is complex and there is a PCR amplification preference, which can easily introduce amplification errors. The advantage of the PCR-free process is that the operation is simpler due to the omission of the PCR process, and it has a more superior sensitivity and accuracy for rare mutation detection than the PCR process because it can avoid the preference and amplification errors brought by PCR amplification. The disadvantage is that it requires a larger sample volume than the PCR process and has a higher index hopping ratio than the PCR process.

Adding a specific index to each sample and uploading it together in the same lane and then separating different sample data during subsequent data analysis is a common method to improve sequencing throughput and avoid instrument waste in second-generation sequencing. The DNA sequence index used to distinguish different samples is called Index. The occurrence of sequence index hopping means that different samples cannot be distinguished correctly based on the index. The increased sequence index hopping ratio in a normal PCR-free library building process means that a larger percentage of the data assigned to an index comes from other samples, and the accuracy of the assay is greatly affected. To solve this problem, dual indexes are generally used to add two indexes to a sample, and only sequences with both indexes correct at the time of data splitting are considered reliable, and sequences with incorrect index combinations are identified and discarded. This method ensures that the split data are determined to be from the same sample and solves the detection accuracy problem caused by sequence index hopping, but increases the difficulty of linker preparation and maintenance due to the increased number of labels.

In addition, redundancy in sequencing platforms using patterned flow cell technology (e.g. NovaSeq™ system) is very high and increases with the amount of sequencing data. Redundancy is the result of a molecule being sequenced multiple times, which is not helpful for data analysis, and therefore redundancy needs to be removed prior to data analysis. Higher redundancy means that more sequencing data is not available and sequencing costs are higher. As an assay with very high data amount requirements, whole genome sequencing is more needed than other assays to reduce sequencing redundancy to save sequencing costs.

### DETAILED DESCRIPTION OF THE DISCLOSURE

In view of the high sequence index hopping ratio and redundancy problems currently encountered in the whole genome sequencing assays, the present application provides a method for constructing a whole genome high-throughput sequencing library that reduces redundancy and sequence index hopping. The method of high-throughput sequencing libraries is applicable to sequencing platforms employing patterned flow cell technology (e.g. NovaSeq™ system), including existing and future sequencing platforms employing patterned flow cell technology.

The present disclosure is based on the following findings:

The present inventors found that sequence index hopping on sequencing platforms based on patterned flow cell technology is caused by residual unengaged index linkers in the library. The more index linkers, the more severe the sequence index hopping (e.g., PCR-free libraries have significantly higher sequence index hopping ratio than PCR libraries. This is because the PCR amplification process of PCR libraries has a dilution effect on the linkers, and the residual amount of index linkers is lower). It is speculated that the principle is that: P7 linker sequence with an index is located at the 3′ end and amplifies after matching the P7 primer on the Flowcell, forming a cluster of primers with the index, which acts as a primer to amplify the template strand after the library template strand is introduced, thus replacing the index carried by the template strand itself, resulting in the sequence index hopping. To solve this problem, the P7 end of the linkers with the index is changed the orientation from the 3′ end to the 5′ end, so that only the P5 strand of the linkers can form a primer cluster with the P5 primer on the flow cell. Because there is no index on P5, and no replacement of the template strand index will occur. Based on this finding, the present inventors made the design of changing the orientation of the linkers to prevent the occurrence of sequence index hopping.

In addition, the present inventors also found that the process of generating clusters when PCR-free libraries are loaded on a sequencing platform using a patterned flow cell may cause the library template strand to fall off due to rapid amplification and fall into an adjacent well to generate another cluster, and this process may happen multiple times. The resulting situation where 1 template is measured twice or more times occurs, which is one of the reasons for the generation of redundancy. The addition of a high GC sequence at the 5′ end of the linkers can hold the template strand during cluster generation and make it less likely to be fallen off. Direct synthesis of such a linker has some problems, mainly because the sequence is too long and difficult to synthesize, and the cost is too expensive. Therefore, a clamp linker containing high GC sequences is designed in the present disclosure. When the Y-shaped linkers are connected, the high GC clamp linkers are connected to the 5′ end of the Y-shaped linker, so as to achieve the effect of adding a section of high GC sequences at the 5′ end of the linker without increasing the operation steps, thus reducing redundancy on the sequencing platforms employing patterned flow cell technology.

Combining the above two findings, the present disclosure combines these two designs to design a novel linker combination of a reverse complementary Y-shaped linker combined with a high GC clamp linker for constructing a whole genome high-throughput sequencing library, thus achieving both reductions of redundancy and sequence index hopping.

Thus, in a first aspect, the present disclosure provides a method for constructing a whole genome high-throughput sequencing library that can reduce redundancy and sequence index hopping, characterized in that the method comprises the steps of:

1) extracting a sample gDNA;

2) fragmenting said sample gDNA by enzyme cleavage, filling ends of the gDNA and adding A base to the gDNA fragments to obtain an A-added gDNA;

3) connecting the A-added gDNA with a linker combination to obtain a connected produce, said linker combination comprises two parts: a Y-shaped reverse linker and a high GC clamp linker;

4) purifying said connected product to obtain a purified product;

5) screening the fragment of said purified product to obtain a sequencing library.

According to a preferred embodiment of the present disclosure, said Y-shaped reverse linker sequence is inversely complementary to a normal Y-shaped linker sequence and has the following sequence:

5′ pCAAGCAGAAGACGGCATACGAGATNNNNNNNGTGACTGGAGTTCAG
ACGTGTGCTCTTCCGAT*C*T 3′
5′ pGATCGGAAGAGCGTCGTGTAGGGAAAGAGTGTAGATCTCGGTGGTC
GCCGTATCATT3′

where N represents randomly degenerate bases A/T/C/G, and for different indexes, different sequences are used, and * represents thio-modification and p represents phosphorylation modification.

According to a preferred embodiment of the present disclosure, said Y-shaped reverse linker is annealed to form the following structure:

5′-CAAGCAGAAGACGGCATACGAGATNNNNNNNGTGACTGGAGTTCAGA
CGTGTGCTCTTCCGATCT-3′
CGAGAAGGCTAG-5′
3′ -TTACTATGCCGCTGGTGGCTCTAGATGTGAGAAAGGGATGTGCTG.

According to a preferred embodiment of the present disclosure, said high GC clamp linker is formed by annealing two sequences: one sequence is a GC clamp sequence, which may be 5-50 bp in length, preferably 11-18 bp in length; the other sequence contains two parts, one part is reverse complementary to the GC clamp sequence and the other part is reverse complementary to the sequence at the P7 end of the Y-shaped reverse linker.

According to a preferred embodiment of the present disclosure, said GC clamp sequences are as follows:

Sequence 1: 5′ TCGACTGCGTG3′
Sequence 2: 5′ CGTATGCCGTCTTCTGCTTGCACGCAGTC3′

The 5′ end of sequence 1, the 5′ end and the 3′ end of sequence 2 are subject to end closure.

According to a preferred embodiment of the present disclosure, said high GC clamp linker is annealed to form a structure as follows:

5′-TCGACTGCGTG-3
3′- CTGACGCACGTTCGTCTTCTGCCGTATGC-5′.

According to a preferred embodiment of the present disclosure, the two parts of said linker combination are annealed and connected together by the principle of base complementarity during the connecting step 3) and then connected to the gDNA fragment in step 2) to form the final library as shown in FIG. 5.

According to a preferred embodiment of the present disclosure, the process for constructing the library described herein requires one purification and one fragment screening.

According to a preferred embodiment of the present disclosure, said high-throughput sequencing method is applicable to sequencing platforms (e.g. NovaSeq™ system, etc.) employing patterned flow cell technology, including existing and future sequencing platforms employing patterned flow cell technology.

According to a preferred embodiment of the present disclosure, said samples may be selected from the group consisting of cell line, peripheral blood, cord blood, amniotic fluid, chorion, placenta, umbilical cord, saliva and pharyngeal swab.

The method for constructing a library of the present disclosure is easy and time consuming to perform.

In a second aspect, the present disclosure provides a kit for constructing a whole genome high-throughput sequencing library that can reduce redundancy and sequence index hopping, said kit comprises the following reagents:

reagents required for fragmenting a sample gDNA, filling ends of the gDNA and adding A base to the gDNA fragments, including enzymes and buffers required for fragmenting, filling ends of the gDNA and adding A base;

connecting reagents, including ligase, ligation buffer and a linker combination required for the connecting step, said linker combination comprises two parts: a Y-shaped reverse linker and a high GC clamp linker; and

reagents and devices required for performing the purification step 4) and the fragment screening step 5).

According to a preferred embodiment of the present disclosure, said Y-shaped reverse linker sequence is inversely complementary to a normal Y-shaped linker sequence and has the following sequence.

5′ pCAAGCAGAAGACGGCATACGAGATNNNNNNNGTGACTGGAGTTCAG
ACGTGTGCTCTTCCGAT*C*T 3′
5′ pGATCGGAAGAGCGTCGTGTAGGGAAAGAGTGTAGATCTCGGTGGTC
GCCGTATCATT3′

where N represents randomly degenerate bases A/T/C/G, and for different indexes, different sequences are used, and * represents thio-modification and p represents phosphorylation modification.

According to a preferred embodiment of the present disclosure, the Y-shaped reverse linker is annealed to form the following structure:

5′ -CAAGCAGAAGACGGCATACGAGATNNNNNNNGTGACTGGAGTTCAG
ACGTGTGCTCTTCCGATCT-3′
CGAGAAGGCTAG-5′
3′ -TTACTATGCCGCTGGTGGCTCTAGATGTGAGAAAGGGATGTGCTG.

According to a preferred embodiment of the present disclosure, said high GC clamp linker is formed by annealing two sequences: one sequence is a GC clamp sequence, which may be 5-50 bp in length, preferably 11-18 bp in length; the other sequence contains two parts, one part is reverse complementary to the GC clamp sequence and the other part is reverse complementary to the sequence at the P7 end of the Y-shaped reverse linker.

According to a preferred embodiment of the present disclosure, said GC clamp sequence is as follows:

Sequence 1: 5′ TCGACTGCGTG3′
Sequence 2: 5′ CGTATGCCGTCTTCTGCTTGCACGCAGTC3′

The 5′ end of sequence 1 and the 5′ and 3′ ends of sequence 2 are subject to end closure.

According to a preferred embodiment of the present disclosure, said high GC clamp linker is annealed to form a structure as follows:

5′-TCGACTGCGTG-3
3′- CTGACGCACGTTCGTCTTCTGCCGTATGC-5′.

According to a preferred embodiment of the present disclosure, said two parts of said linker combination are annealed and connected together by the principle of base complementarity during the connecting step and then connected to said gDNA fragment to form the final library.

According to a preferred embodiment of the present disclosure, said kit is applicable to sequencing platforms employing patterned flow cell technology (e.g. NovaSeq™ system), including existing and future sequencing platforms employing patterned flow cell technology.

According to a preferred embodiment of the present disclosure, said sample may be selected from the group consisting of a cell line, peripheral blood, cord blood, amniotic fluid, chorion, placenta, umbilical cord, saliva and pharyngeal swab.

In a third aspect, the present disclosure provides a Y-shaped linker characterized in that said Y-shaped reverse linker sequence is inversely complementary to a normal Y-shaped linker sequence. According to a preferred embodiment of the present disclosure, the sequence of said Y-shaped reverse linker is as follows:

5′ pCAAGCAGAAGACGGCATACGAGATNNNNNNNGTGACTGGAGTTCAG
ACGTGTGCTCTTCCGAT*C*T 3′
5′ pGATCGGAAGAGCGTCGTGTAGGGAAAGAGTGTAGATCTCGGTGGTC
GCCGTATCATT3′

where N represents random degenerate base A/T/C/G and * represents thio-modification and p represents phosphorylation modification.

According to a preferred embodiment of the present disclosure, said Y-shaped reverse linker is annealed to form the following structure:

5′ -CAAGCAGAAGACGGCATACGAGATNNNNNNNGTGACTGGAGTTCAG
ACGTGTGCTCTTCCGATCT-3′
CGAGAAGGCTAG-5′
3′ -TTACTATGCCGCTGGTGGCTCTAGATGTGAGAAAGGGATGTGCTG.

In a fourth aspect, the present disclosure provides a high GC clamp linker, characterized in that said high GC clamp linker is formed by annealing two sequences: one sequence is a GC clamp sequence, which may be 5-50 bp in length, preferably 11-18 bp in length; the other sequence contains two parts, one part is reverse-complementary to the GC clamp sequence and the other part is reverse-complementary to the sequence at the P7 end of the Y-shaped reverse linker.

According to a preferred embodiment of the present disclosure, said GC clamp sequence is as follows.

Sequence 1: 5′ TCGACTGCGTG3′
Sequence 2: 5′ CGTATGCCGTCTTCTGCTTGCACGCAGTC3′.

The 5′ end of sequence 1 and the 5′ and 3′ ends of sequence 2 are subject to end closure.

According to a preferred embodiment of the present disclosure, said high GC clamp linker is annealed to form a structure as follows:

5′-TCGACTGCGTG-3′
3′-CTGACGCACGTTCGTCTTCTGCCGTATGC-5′

In a fifth aspect, the present disclosure provides a linker combination characterized in that it comprises the Y-shaped reverse linker as described above and the high GC clamp linker as described above.

According to a preferred embodiment of the present disclosure, the two components of said novel linker combination are annealed and connected together by the principle of base complementarity during the connecting step and then connected to the gDNA fragment to form the final library.

As used in the present disclosure, the term “reverse complementary” means that, as in the case of a high GC clamp linker, a part of one sequence: 5′-GACTGCGTG-3′ and a part of another sequence 3′-CTGACGCAC-5′ are in opposite directions (5′ to 3′ in one direction and 3′ to 5′ in the other), and the sequences are complementary (base pairing principle, i.e. adenine A pairs with thymine T and guanine G pairs with cytosine C), i.e., the two sequences are reverse complementary to each other.

A part of sequences in the high GC clamp linker of the present disclosure: 3′-GTTCGTCTTCTGCCGTATGC-5′ and a part of sequences in the Y-shaped reverse complementary linker: 5′-CAAGCAGAAGACGGCATACG-3′ are also reverse complementary sequences to each other, and the high GC clamp linker and the Y-shaped reverse complementary linker are connected together by the part of reverse complementary sequence under the principle of base complementary pairing and the action of ligase to form the novel linker combination described in the present disclosure.

In one embodiment, a whole genome high-throughput sequencing library is constructed using a common Y-shaped linker (TrueSeq linker), a Y-shaped reverse linker, and a novel linker combination (high GC clamp linker with Y-shape reverse linker), respectively, and sequenced on NovaSeq platform. The data analysis and comparison confirmed that the library constructed using the novel linker combination had the lowest redundancy, indicating that the novel linker combination could effectively reduce redundancy.

In another embodiment, a human genomic DNA is used and PCR-free libraries are constructed using Y-shaped linker (TrueSeq linker) and the novel linker combination, respectively, and sequenced together with phix libraries. The number of sequences of phix measured in the libraries is analyzed to calculate the index hopping ratio. The phix sequences are not present on the human genome. So normally the phix library sequences are not detected in the libraries constructed from the human genome. Only when the sequence index hopping occurs, the phix sequence carries the above-mentioned index of the human genome library and will be detected in the human genome library when the data is split according to the index during data analysis. In other words, the percentage of phix sequences split from the human genome library reflects the sequence index hopping ratio of that library. Therefore, the sequence index hopping ratio can be obtained by calculating the proportion of the actual number of detected phix library sequences to the total library sequence number. It was found that the sequence index hopping ratio in the library constructed with the novel linker combination was significantly lower than that in the library constructed with the common Y-shaped linker, indicating that the novel linker combination could effectively reduce the sequence index hopping situation.

In one embodiment, different DNA input amounts are tested, and it is found that when the input amount is less than 300 ng, although there is little difference in quality control analysis, there will be some effect on the performance analysis. This is probably because when the DNA input amount is insufficient, the abundance of its library is low, thus affecting the accuracy of the performance analysis results. So it is necessary to ensure at least 300 ng input amount.

In one embodiment, the quality control analysis and performance analysis results of the same library measured with different data amounts are tested for comparison. It is found that when the data amount is too small, neither the quality control results nor the performance analysis results can meet the analysis requirements. As the data amount increased to a certain level, the quality control results and performance analysis results are no longer significantly improved. Therefore, an optimal amount of sequencing data can be determined that does not cause waste and can meet the analytical requirements.

Compared with the currently available methods for constructing whole genome high-throughput sequencing libraries, the present disclosure adopts a PCR-Free approach to construct libraries, which can reduce the preference generated by amplification. The novel linker combination of the present disclosure effectively reduces sequence index hopping and redundancy on NovaSeq platform, which saves sequencing cost. Moreover, the present disclosure can complete the library construction in one tube, which is easy to operate and greatly shortens the library construction time.

The present disclosure will be described in detail below with reference to the drawings and in conjunction with embodiments. It should be noted that those skilled in the art should understand that the drawings of the present disclosure and the embodiments thereof are for the purpose of illustration only and do not constitute any limitation to the present disclosure. Without contradiction, the embodiments and the features in the embodiments of the present application may be combined with each other.

### DESCRIPTION OF THE FIGURES

FIG. 1 shows a flowchart of a method for constructing a whole genome high-throughput sequencing library according to the present disclosure.

FIG. 2 shows the structure of a normal Y-shaped linker (TruSeq linker), consisting of P7, P5 sequences, index sequences and sequencing primer sequences (R1 SP and R2 SP).

FIG. 3 shows the structure of a Y-shaped reverse linker according to the present disclosure, consisting of the reverse complementary P7, P5 sequences, index sequences and sequencing primer sequences (R1 SP and R2 SP).

FIG. 4 shows a novel clamp linker combination according to the present disclosure, consisting of a Y-shaped reverse complementary linker and a high GC clamp linker.

FIG. 5 shows a whole genome high-throughput sequencing library structure according to the present disclosure, wherein a fragmented, end-filled and A-added gDNA is connected to a novel linker combination to form a second generation sequencing library.

FIG. 6 shows the distribution of insert fragments of DNA libraries with different input amounts in accordance with example 3 of the present disclosure.

FIG. 7 shows the distribution of sequencing depths and densities of DNA libraries with different input amounts in accordance with example 3 of the present disclosure.

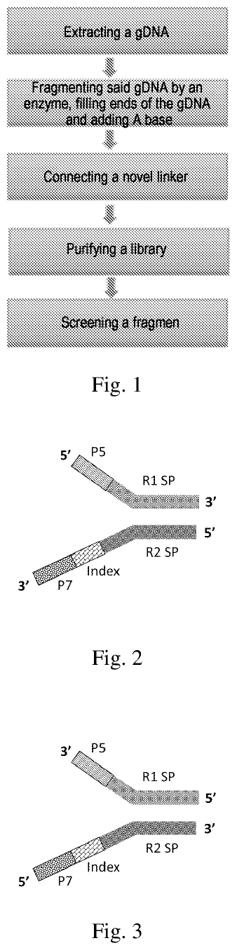

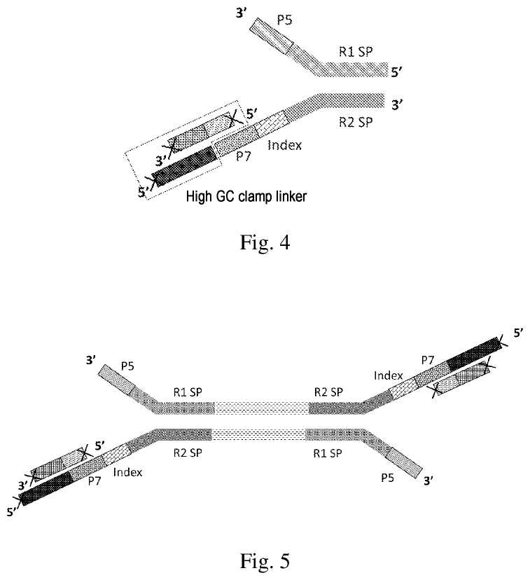

### EXAMPLES

The present disclosure will be described in detail below with reference to the drawings and in conjunction with examples.

The specific sequence of the common Y-shaped linker (TruSeqlinker) used in the following example is as follows.

5′ AATGATACGGCGACCACCGAGATCTACACTCTTTCCCT
ACACGACGCTCTTCCGAT*C*T3′
5′ pGATCGGAAGAGCACGTCTGAACTCCAGTCACNNNNNNNNATCTCGT
ATGCCGTCTTCTGCTTG 3′

where N represents randomly degenerate bases A/T/C/G and * represents thio-modification, and p represents phosphorylation modification.

### Example 1

The standard cell line NA12878 genomic DNA was used to construct PCR-free libraries by using a normal Y-shaped linker (TruSeq linker), a Y-shaped reverse linker according to the disclosure, and a novel linker combination according to the disclosure (Y-shaped reverse linker+high GC clamp linker), respectively, and the PCR library was used as a control. The sequencing data of the PCR-free library constructed by the three different linkers and the PCR library were compared by sequencing and data analysis.

NA12878 gDNA as the sample was used in the example, and PCR-free whole genome high-throughput sequencing libraries were constructed using the normal Y-shaped linker, the Y-shaped reverse linker according to the disclosure, and the Y-shaped reverse linker+high GC clamp linker according to the disclosure, respectively. The libraries were subjected to 150PE double-end sequencing on NovaSeq, and the sequencing results were analyzed using bioinformatics. The following was the specific protocol.

Step 1: A reaction mixture as shown in Table 1 was prepared. 3 tubes were prepared for subsequent connection of 3 different linkers. Then, the reaction procedures of fragmentation, end-filling and addition of A base as shown in Table 2 were run together.

TABLE 1

| Component | Volume | |
|---|---|---|
| NA12878 gDNA 300 ng | X | μl |
| WGS reactive enzyme f | 5 | μl |
| WGS buffer f | 2.5 | μl |
| Sterile H₂O | 17.5-X | μl |
| Total volume | 25 | μl |

TABLE 2

| Reaction | Reaction |
|---|---|
| temperature | time |
| 4° C. | 1 min |
| 32° C. | 6 min |
| 65° C. | 30 min |
| 4° C. | ∞ |

Hot cap temperature: 70° C., volume: 25 μl.

Step 2: Each reaction component needed for connecting as shown in Table 3 for 1, 2 and 3 was added to the reaction solutions of fragmentation, end-filling and addition of A base of Step 1 respectively, and the connecting procedure was run as shown in Table 4.

TABLE 3

| Component | 1 | 2 | 3 |
|---|---|---|---|
| Previous reaction system | 25 μl | 25 μl | 25 μl |
| WGS connecting solution | 10 μl | 10 μl | 10 μl |
| WGS ligase | 5 μl | 5 μl | 5 μl |
| WGS normal Y-shaped linker (3 μM) | 5 μl | — | — |
| WGS Y-shaped reverse linker (3 μM) | — | 5 μl | 5 μl |
| WGS high GC clamp linker (3 μM) | — | — | 5 μl |
| Sterile H₂O | 5 μl | 5 μl | — |
| Total volume | 50 μl | 50 μl | 50 μl |

TABLE 4

| Reaction | Reaction |
|---|---|
| temperature | time |
| 20° C. | 15 min |
| 4° C. | ∞ |

Step 3: The amplification products were purified using the High-throughput Sequencing Library Construction DNA Purification Kit (magnetic bead method).

Step 4: The purified libraries were screened for fragments using the High-throughput Sequencing Library Construction DNA Purification Kit (magnetic bead method), wherein 0.49× beads were added for binding, the beads were discarded, the supernatant was removed, and 0.15× beads were further added for binding, washing and elution.

Step 5: The screened libraries were purified for qPCR quantification.

Step 6: Based on the qPCR quantification results, the libraries were sequenced by NovaSeq 150PE double-end sequencing according to the standard operating procedures of the sequencer.

Step 7: The sequencing results were subjected to basic statistics and performance analysis, and the basic statistics are shown in Table 5.

TABLE 5

| | Type | | | |
|---|---|---|---|---|
| | | | | Y-shaped |
| | | normal Y- | Y-shaped | reverse linker + |
| | | shaped | reverse | high GC |
| | | linker | linker | clamp linker |
| | Sample Name | | | |
| | Control-PCR | RWGSNA | RWGSNA | RWGSNA |
| | NA12878 | 12878A2 | 12878R2A | 12878GC11rep2 |
| Number of data | 722.6 | 722.6 | 722.6 | 722.6 |
| amount | | | | |
| sequences: M | | | | |
| Comparison | 94.54 | 94.06 | 94.24 | 95.50 |
| ratio % | | | | |
| Redundancy % | 15.08 | 28.81 | 18.12 | 4.94 |
| Average | 29.88 | 21.94 | 26.01 | 30.04 |
| sequencing depth | | | | |
| Coverage % | 98.10 | 98.11 | 98.18 | 98.20 |
| 10× coverage % | 97.09 | 96.90 | 97.31 | 97.44 |
| 20× coverage % | 92.68 | 65.85 | 85.83 | 92.55 |
| Insert fragment | 370 | 286 | 316 | 309 |
| size | | | | |

Note:
The first column NA12878 in Table 5 is the PCR process control, and the samples and volumes are consistent with this example, and the library was constructed using the PCR amplification method as a control. The analysis results showed that the redundancy of the library constructed according to the novel linker combination of the present disclosure (4.78%) was significantly lower than those of the other libraries (14.69%, 27.6%, and 18.25%) under the same amount of data. Because its redundancy was significantly lower than those of the other three libraries, its average sequencing depth was deeper and the 20× coverage was higher, which was comparable to the performance of the library constructed by the PCR process. It indicates that the use of the novel linker combination according to the present disclosure effectively reduces the redundancy of the PCR-free library and the overall quality control data performs optimally.

TABLE 6

| | | SNP | | INDEL | | CNV | | Repeat |
|---|---|---|---|---|---|---|---|---|
| | | Accuracy | Sensitivity | Accuracy | Sensitivity | Accuracy | Sensitivity | Consistency |
| Type | Sample | % | % | % | % | % | % | % |
| Control- | NA12878 | 99.54 | 98.96 | 94.90 | 94.3 | 51.1 | 96.6 | 78.1 |
| PCR | | | | | | | | |
| Normal | RWGSNA12878A2 | 99.28 | 98.86 | 96.86 | 95.45 | 49.45 | 91.06 | 93.75 |
| Y- | | | | | | | | |
| shaped | | | | | | | | |
| linker | | | | | | | | |
| Y- | RWGSNA12878R2A | 99.37 | 99.13 | 97.76 | 97.33 | 48.07 | 91.38 | 87.50 |
| shaped | | | | | | | | |
| reverse | | | | | | | | |
| linker | | | | | | | | |
| Y- | RWGSNA12878GC11rep2 | 99.27 | 99.22 | 97.37 | 97.49 | 51.30 | 97.10 | 96.90 |
| shaped | | | | | | | | |
| reverse | | | | | | | | |
| linker + | | | | | | | | |
| high | | | | | | | | |
| GC | | | | | | | | |
| clamp | | | | | | | | |
| linker | | | | | | | | |

The results of the performance analysis are shown in Table 6. The accuracy of the novel linker combination of SNPs was comparable to the performance of other libraries and PCR controls, and the sensitivity of the novel linker combination was comparable to the results of Y-shaped reverse linkers and slightly higher than that of normal Y-shaped linker libraries and PCR libraries. As for the accuracy and sensitivity of INDEL, the novel linker combination according to the present disclosure was comparable to the performance of Y-shaped reverse linkers and both were higher than that of normal Y-shaped reverse linkers library and PCR controls. The accuracy of CNV of the novel linker combination library according to the disclosure was comparable to that of the PCR library, higher than those of the normal Y-shaped linker library and the Y-shaped reverse linker library, and the sensitivity was also higher than those of the other three libraries. The concordance rate of repeat was also significantly higher than those of the other three libraries. The results showed that the overall data performance of the libraries constructed with the novel linker combination was better than those of the other three linker libraries in the performance analysis.

### Example 2

The human genomic DNA was used to construct PCR-free libraries using normal Y-shaped linker (TrueSeq linker), Y-shaped reverse linker, and the combination of normal Y-shaped linker and high GC clamp linker and the novely linker combination of the present disclosure, respectively. The libraries were sequenced together with the phix library. The number of phix sequences measured in the library was analyzed, and the index hopping ratio was calculated. Meanwhile, the redundancy under the same amount of data was compared

The principle for testing the hopping ratio using phix is: the phix library insert fragment is derived from viral genomic DNA. Its gene sequences are known precisely and the GC ratio is about 40, which is close to the GC ratio of the human genome. Its gene sequence is far from the human gene sequence and does not contain an index. Therefore, the phix library is sequenced together with the library to be tested, and the number of phix sequences split in the library is analyzed. The ratio of phix sequences to the total number of sequences in the library is calculated as the hopping ratio. The following is the specific protocol.

Step 1: PCR-Free library was constructed with the four linkers respectively.

Step 2: Based on the qPCR quantification results, the phix library and the PCR-free library constructed with four different linkers were sequenced together in 150PE double-end sequencing according to the sequencer standard operation protocol.

Step 3: The sequencing results were compared with the human genome reference sequence and phix gene sequence, and the number of sequences aligned to the human genome reference sequence and the number of sequences aligned to the phix gene sequence were counted. The statistical results are shown in Table 7 below.

TABLE 7

| | | | Number of | | | |
|---|---|---|---|---|---|---|
| | | | Phix | | | |
| Total | | | sequences | | | |
| number of | | Number of | measured | | | |
| Phix | Sample | library | in the | Hopping | | |
| sequences | name | sequences | library | ratio | Redundancy | Remarks |
| 44687582 | RDWGS-304 | 28774642 | 993 | 0.0035% | 9.80% | Normal Y- |
| | | | | | | shaped linker |
| | RDWGS-347R | 40284997 | 473 | 0.0012% | 7.93% | Y-shaped |
| | | | | | | reverse linker |
| | RDWGS-305GC | 19890566 | 2261 | 0.0114% | 5.44% | Normal Y- |
| | | | | | | shaped |
| | | | | | | linker + high |
| | | | | | | GC clamp |
| | | | | | | linker |
| | RDWGS-348RGC | 35998134 | 1161 | 0.0032% | 3.09% | Y-shaped |
| | | | | | | reverse |
| | | | | | | linker + high |
| | | | | | | GC clamp |
| | | | | | | linker |

The results showed that the hopping ratio of the Y-shaped linker was lower than that of the normal Y-shaped linker library. Meanwhile, the hopping ratio of the PCR-free library constructed with the Y-shaped linker+high GC clamp linker was lower than that of the normal Y-shaped linker+high GC clamp linker. Regardless of the addition of high GC clamp linker or not, PCR libraries using Y-shaped reverse linkers showed a lower index hopping ratio, indicating that the specific structure of Y-shaped reverse linkers can effectively reduce the index hopping ratio. Whether combined with the normal Y-shaped linker or the Y-shaped reverse linker, the high GC clamp linker can effectively reduce the redundancy.

### Example 3

The library was constructed using different template input amounts, sequenced, and the data was analyzed to compare the sequencing data quality and the performance analysis results of different input amounts.

The novel linker combination described according to the present disclosure was used in the example to construct whole genome high-throughput sequencing libraries with input amounts of 200 ng and 300 ng respectively, using NA12878 genomic DNA as the sample. The libraries were sequenced by 150PE double-end sequencing on NovaSeq, and the sequencing results were analyzed using bioinformatics to analyze the library quality of libraries constructed with different linkers. The following is the specific scheme.

Step 1: A reaction mixture was prepared as shown in Table 8. Four tubes were prepared, two with 200 ng DNA input amount and the other two with 300 ng DNA input amount. Then, the reaction procedures of fragmentation, end-filling and addition of A base were run together as shown in Table 9.

TABLE 8

| Component | 200 ng | | 300 ng | |
|---|---|---|---|---|
| NA12878 gDNA | X | μl | Y | μl |
| WGS Reactive Enzyme f | 5 | μl | 5 | μl |
| WGS buffer f | 2.5 | μl | 2.5 | μl |
| Sterile H₂O | 17.5-X | μl | 17.5-Y | μl |
| Total volume | 25 | μl | 25 | μl |

TABLE 9

| Reaction | Reaction |
|---|---|
| temperature | time |
| 4° C. | 1 min |
| 32° C. | 6 min |
| 65° C. | 30 min |
| 4° C. | ∞ |

Hot cap temperature: 70° C., volume: 25 μl.

Step 2: Each reaction component needed for connecting as shown in Table 10 was added to the reaction solutions of fragmentation, end-filling and addition of A base of step 1 respectively, and the connecting procedure was run as shown in Table 11.

TABLE 10

| Component | Volume |
|---|---|
| Previous reaction system | 25 μl |
| WGS connecting solution | 10 μl |
| WGS ligase | 5 μl |
| WGS Y-shaped reverse linker (3 μM) | 5 μl |
| WGS high GC clamp linker (3 μM) | 5 μl |
| Total volume | 50 μl |

TABLE 11

| Reaction | Reaction |
|---|---|
| temperature | time |
| 20° C. | 15 min |
| 4° C. | ∞ |

Hot cap temperature: off; volume: 50 μl

Step 3: The amplification products were purified using the High-throughput Sequencing Library Construction DNA Purification Kit (magnetic bead method).

Step 4: The purified libraries were screened for fragments using the High-throughput Sequencing Library Construction DNA Purification Kit (magnetic bead method), wherein the screening conditions are: after binding with 0.49× beads, the beads were discarded, the supernatant was removed, and 0.15× beads were further added for binding, washing and elution.

Step 5: The screened libraries were purified for qPCR quantification.

Step 6: Based on the qPCR quantification results, the libraries were sequenced by NovaSeq 150PE double-end sequencing according to the standard operating procedures of the sequencer.

Step 7: The sequencing results are subjected to basic statistics as well as performance analysis, and the basic statistics are shown in Table 12.

TABLE 12

| | Type | | | |
|---|---|---|---|---|
| | 200 ng | | 300 ng | |
| | Sample Name | | | |
| | NA12878-1 | NA12878-3 | NA12878-5 | NA12878-7 |
| Number of data amount sequences: M | 799.3 | 799.2 | 799.4 | 799.4 |
| Comparison ratio % | 99.50 | 99.47 | 99.69 | 99.70 |
| Redundancy % | 5.91 | 5.18 | 7.53 | 7.29 |
| Average sequencing depth | 33.59 | 34.29 | 33.65 | 33.36 |
| Coverage % | 99.22 | 99.22 | 99.22 | 99.22 |
| 4× Coverage % | 99.11 | 99.11 | 99.12 | 99.12 |
| 20× Coverage % | 96.62 | 96.97 | 96.74 | 96.54 |
| Insert fragment size | 290 | 300 | 303 | 292 |

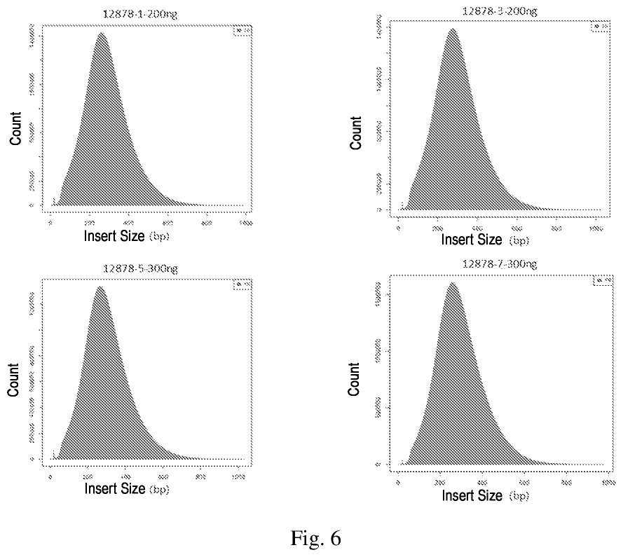

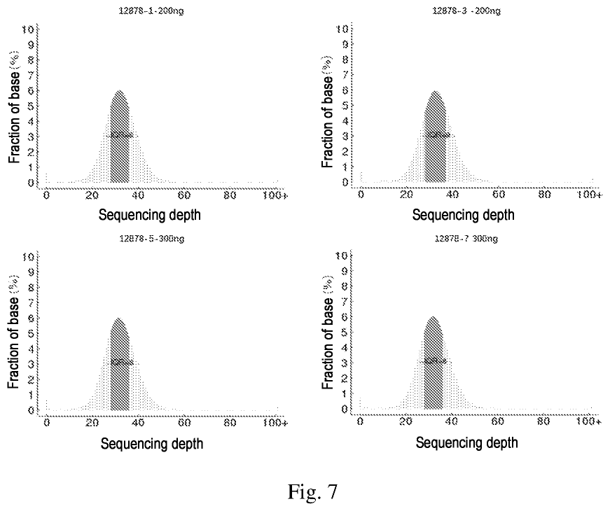

The analysis results show that the quality of the library with 200 ng input amount is comparable to that of the library with 300 ng input amount in terms of basic statistics. FIG. 6 and FIG. 7 show the insert fragment distribution and depth and density distribution, respectively. There is no significant difference in the insert fragment size and depth and density distribution of the library. In the insert fragment distribution, the horizontal coordinate is the fragment size (bp) and the vertical coordinate is the count, which reflects the size distribution of DNA fragments in the library. In the depth and density distribution, the horizontal coordinate is sequencing depth and the vertical coordinate is the count, which reflects the uniformity of sequencing. The narrower the peak, the closer the sequencing depth at each position, i.e., the more uniform the data coverage across the genome, it will be more beneficial for detection of the mutation and CNV.

TABLE 13

| | | SNP | | INDEL | | CNV | | Repeat |
|---|---|---|---|---|---|---|---|---|
| | | Accuracy | Sensitivity | Accuracy | Sensitivity | Accuracy | Sensitivity | Consistency |
| Type | Sample | % | % | % | % | % | % | % |
| 200 | 12878-1 | 99.30 | 99.18 | 97.12 | 96.75 | 24.52 | 96.90 | 90.63 |
| ng | 12878-3 | 99.32 | 99.20 | 97.23 | 96.91 | 17.45 | 96.90 | 93.75 |
| 300 | 12878-5 | 99.33 | 99.22 | 97.51 | 97.47 | 53.61 | 97.39 | 96.88 |
| ng | 12878-7 | 99.20 | 99.20 | 97.31 | 97.24 | 53.57 | 97.39 | 93.75 |

The results of the performance analysis are shown in Table 13. The sensitivity and accuracy data of the two different input amounts of SNP & INDEL performed comparably, the sensitivity of CNV also performed comparably, and the performance of repeat results was also basically comparable. However, the accuracy of 300 ng input amount of CNV was significantly higher than that of 200 ng.

### Example 4

The whole genome high-throughput sequencing library was constructed, sequenced, and the data amounts of 15×, 30× and 40× sequencing depths were intercepted. Data analysis, basic statistics and performance analysis were performed to compare the data performance under different sequencing depths.

The novel linker combination described according to the present disclosure was used in the example to construct whole genome high-throughput sequencing libraries with input amount of 300 ng, using NA12878 genomic DNA as the sample. The libraries were sequenced by 150PE double-end sequencing on NovaSeq, and the sequencing results were analyzed using bioinformatics to analyze the library quality of libraries constructed with different linkers. The following is the specific scheme.

Step 1: A reaction mixture was prepared as shown in Table 14. Then, the reaction procedures of fragmentation, end-filling and addition of A base as shown in Table 2 were run.

TABLE 14

| Component | Volume | |
|---|---|---|
| NA12878 gDNA | X | μl |
| WGS Reactive Enzyme f | 5 | μl |
| WGS buffer f | 2.5 | μl |
| Sterile H₂O | 17.5-X | μl |
| Total volume | 25 | μl |

TABLE 15

| Reaction | Reaction |
|---|---|
| temperature | time |
| 4° C. | 1 min |
| 32° C. | 6 min |
| 65° C. | 30 min |
| 4° C. | ∞ |

Hot cap temperature: 70° C., volume: 25 μl

Step 2: Each reaction component needed for connecting as shown in Table 16 was added to the reaction solutions of fragmentation, end-filling and addition of A base of step 1 respectively, and the connecting procedure was run as shown in Table 17.

TABLE 16

| Component | Volume |
|---|---|
| Previous reaction system | 25 μl |
| WGS connecting solution | 10 μl |
| WGS ligase | 5 μl |
| WGS Y-shaped reverse linker (3 μM) | 5 μl |
| WGS high GC clamp linker (3 μM) | 5 μl |
| Total volume | 50 μl |

TABLE 17

| Reaction | Reaction |
|---|---|
| temperature | time |
| 20° C. | 15 min |
| 4° C. | ∞ |

Hot cap temperature: off; volume: 50 μl

Step 3: The amplification products were purified using the High-throughput Sequencing Library Construction DNA Purification Kit (magnetic bead method).

Step 4: The purified libraries were screened for fragments using the High-throughput Sequencing Library Construction DNA Purification Kit (magnetic bead method), wherein the screening conditions are: 0.49× beads were added for binding, the supernatant was taken, and 0.15× beads were further added for binding, washing and elution.

Step 5: The screened libraries were purified for qPCR quantification.

Step 6: Based on the qPCR quantification results, the libraries were subjected to NovaSeq 150PE double-end sequencing according to the standard operating procedures of the sequencer.

Step 7: The sequencing results were theoretically calculated and the data amounts were intercepted from the beginning of 15×, 30× and 40× data respectively for basic statistical analysis. (Note: there will be some deviation between the theoretically calculated data amounts and the actual intercepted data amounts. According to the theoretical calculation, 15×, 30×, and 40× data amounts are intercepted, but the actual intercepted data amounts are 17×, 33×, and 38×, respectively). The results are shown in Table 18.

TABLE 18

| | Depth | | | | | | | | | | | |
|---|---|---|---|---|---|---|---|---|---|---|---|---|
| | 17× | | | | 33× | | | | 38× | | | |
| | Sample | | | | | | | | | | | |
| | 5 | 6 | 7 | 8 | 5 | 6 | 7 | 8 | 5 | 6 | 7 | 8 |
| Number of | 400 | 400 | 400 | 400 | 799 | 799 | 799 | 799 | 933 | 933 | 933 | 933 |
| data amount | | | | | | | | | | | | |
| sequence: M | | | | | | | | | | | | |
| Comparison | 99.70 | 99.71 | 99.72 | 99.68 | 99.69 | 99.70 | 99.70 | 99.68 | 99.68 | 99.69 | 99.70 | 99.67 |
| rate % | | | | | | | | | | | | |
| Average | 16.44 | 16.92 | 16.37 | 33.65 | 32.48 | 33.36 | 32.44 | 39.10 | 37.74 | 38.77 | 37.75 | 16.44 |
| sequencing | | | | | | | | | | | | |
| depth | | | | | | | | | | | | |
| Coverage % | 99.18 | 99.18 | 99.18 | 99.18 | 99.22 | 99.22 | 99.22 | 99.22 | 99.23 | 99.23 | 99.23 | 99.23 |
| 4 × | 99.0 | 99.0 | 99.0 | 98.9 | 99.1 | 99.1 | 99.1 | 99.1 | 99.1 | 99.1 | 99.1 | 99.1 |
| coverage % | | | | | | | | | | | | |
| 20 × | 23.4 | 19.9 | 23.0 | 19.9 | 96.7 | 95.8 | 96.5 | 95.5 | 98.2 | 98.0 | 98.2 | 97.9 |
| coverage % | | | | | | | | | | | | |
| Redundancy | 6.5 | 6.1 | 5.9 | 5.7 | 7.5 | 7.2 | 7.3 | 6.5 | 8.0 | 7.7 | 7.8 | 6.9 |
| % | | | | | | | | | | | | |
| Insert size | 303 | 276 | 292 | 271 | 303 | 276 | 292 | 271 | 303 | 276 | 292 | 271 |

The analysis results show that the coverage of 20× increases with the increase of sequencing depth. When the sequencing depth is 17×, the coverage of 20× is only 20-23%; and the other QC points data amount, average sequencing depth and redundancy will be slightly improved with the increase of sequencing depth. Overall, the basic statistics of 17× are poor, while the basic statistics of 33× and 38× are comparable.

| | | SNP | | INDEL | | CNV | | Repeat |
|---|---|---|---|---|---|---|---|---|
| | | Accuracy | Sensitivity | Accuracy | Sensitivity | Accuracy | Sensitivity | Consistency |
| Type | Sample | % | % | % | % | % | % | % |
| 17× | NA12878-5 | 99.15 | 97.18 | 94.39 | 90.09 | 60.4 | 90.9 | 87.5 |
| | NA12878-6 | 98.99 | 96.93 | 94.12 | 89.43 | 60.5 | 88.2 | 87.5 |
| | NA12878-7 | 99.11 | 97.23 | 94.41 | 90.22 | 60.6 | 91.8 | 71.9 |
| | NA12878-8 | 98.96 | 96.9 | 94.08 | 89.3 | 61.8 | 91.7 | 87.5 |
| 33× | NA12878-5 | 99.33 | 99.22 | 97.51 | 97.47 | 53.6 | 97.4 | 96.9 |
| | NA12878-6 | 99.22 | 99.21 | 97.30 | 97.23 | 54.3 | 96.6 | 90.6 |
| | NA12878-7 | 99.30 | 99.23 | 97.45 | 97.42 | 53.6 | 97.4 | 90.6 |
| | NA12878-8 | 99.20 | 99.20 | 97.31 | 97.24 | 55.0 | 96.4 | 93.8 |
| 38× | NA12878-5 | 99.34 | 99.3 | 99.82 | 95.24 | 53.0 | 97.4 | 96.9 |
| | NA12878-6 | 99.25 | 99.29 | 99.79 | 94.99 | 53.8 | 96.6 | 90.6 |
| | NA12878-7 | 99.33 | 99.3 | 99.82 | 94.66 | 53.1 | 97.7 | 90.6 |
| | NA12878-8 | 99.23 | 99.29 | 99.79 | 94.87 | 54.4 | 97.2 | 93.8 |

The results of the performance analysis are shown in Table 19. The accuracy and sensitivity of SNPs performed comparably at different sequencing depths, the accuracy and sensitivity of INDEL were lower at 17× than at 33×, while performed comparably at 33× and 38×. The accuracy of CNV was higher at 17× than at 33×, and performed comparably at 33× and 38×. The sensitivity of CNV was lower at 17× than at 33×, and performed comparably at 33× and 38×. The consistency of repeat was lower at 17× than at 33×, and performed comparably at 33× and 38×. Overall, the performance analysis at 17× was a little worse than at 33× and could not meet the analysis demand, while the performance analysis at 38× was basically comparable to that at 33×. Considering the sequencing results as well as the cost, the sequencing depth of 33× is the optimal sequencing depth.

1. A method for constructing a whole genome high-throughput sequencing library, comprising the steps of:
1) extracting a sample gDNA;
2) fragmenting said sample gDNA by enzyme cleavage, filling ends of the gDNA and adding A base to the gDNA fragments to obtain an A-added gDNA;
3) connecting the A-added gDNA with a linker combination to obtain a connected product, said linker combination comprises two parts: a Y-shaped reverse linker and a high GC clamp linker;
4) purifying said connected product to obtain a purified product;
5) screening the fragment of said purified product to obtain a sequencing library.

2. The method according to claim 1, characterized in that said Y-shaped reverse linker sequence is reverse complementary to a normal Y-shaped linker sequence and has the following sequence:
5′pCAAGCAGAAGACGGCATACGAGATNNNNNNNGTGACTGGAGTTCAGA
CGTGTGCTCTTCCGAT*C*T 3′
5′pGATCGGAAGAGCGTCGTGTAGGGAAAGAGTGTAGATCTCGGTGGTCG
CCGTATCATT3′
wherein N represents random degenerate base A/T/C/G, * represents thio-modification and p represents phosphorylation modification.

3. The method according to claim 1, characterized in that said Y-shaped reverse linker is annealed to form the structure of:
5′ -CAAGCAGAAGACGGCATACGAGATNNNNNNNGTGACTGGAGTTCAG
ACGTGTGCTCTTCCGATCT-3′
CGAGAAGGCTAG-5′
3′ -TTACTATGCCGCTGGTGGCTCTAGATGTGAGAAAGGGATGTGCTG′.

4. The method according to claim 1, characterized in that said high GC clamp linker is formed by annealing two sequences: one sequence is a GC clamp sequence, which is 5-50 bp in length; the other sequence contains two parts, one part is reverse complementary to the GC clamp sequence and the other part is reverse complementary to the sequence at the P7 end of the Y-shaped reverse linker.

5. The method according to claim 4, characterized in that said GC clamp sequences are as follows.
Sequence 1: 5′ TCGACTGCGTG3′
Sequence 2: 5′ CGTATGCCGTCTTCTGCTTGCACGCAGTC3′
wherein the 5′ end of sequence 1, the 5′ end and the 3′ end of sequence 2 are end closed.

6. The method according to claim 5, characterized in said high GC clamp linker is annealed to form the structure of:
5′-TCGACTGCGTG-3′
3′-CTGACGCACGTTCGTCTTCTGLCGTATGC-5′.

7. The method according to claim 1, characterized in that
(a) said two parts of said linker combination are annealed and connected together by the principle of base complementarity during the connecting step 3) and then connected to the gDNA fragment in step 2) to form the final library; or
(b) said method is applicable to a sequencing platform employing patterned flow-through technology; or
(c) said sample is selected from the group consisting of a cell line, peripheral blood, cord blood, amniotic fluid, chorion, placenta, umbilical cord, saliva and pharyngeal swab.

8. (canceled)

9. (canceled)

10. A kit for constructing a whole genome high-throughput sequencing library, characterized in that it comprises the following components:
reagents required for fragmenting a sample gDNA, filling ends of the gDNA and adding A base, including enzymes and buffers required for fragmenting, filling ends of the gDNA and adding A base;
connecting reagents, including ligase, ligation buffer and a linker combination required for the connecting step, said linker combination comprises two parts: a Y-shaped reverse linker and a high GC clamp linker; and
reagents and devices required for purifying a connected product to obtain a purified product, and for screening a fragment of the purified product to obtain a sequencing library.

11. The kit according to claim 10, characterized in that said Y-shaped reverse linker sequence is reverse-complementary to a normal Y-shaped linker sequence and has the following sequence:
5′pCAAGCAGAAGACGGCATACGAGATNNNNNNNGTGACTGGAGTTCAGA
CGTGTGCTCTTCCGAT*C*T 3′
5′pGATCGGAAGAGCGTCGTGTAGGGAAAGAGTGTAGATCTCGGTGGTCG
CCGTATCATT3′
where N represents random degenerate base A/T/C/G, * represents thio-modification and p represents phosphorylation modification.

12. The kit according to claim 10, characterized in that the Y-shaped reverse linker is annealed to form the following structure:
5′ -CAAGCAGAAGACGGCATACGAGATNNNNNNNGTGACTGGAGTTCAG
ACGTGTGCTCTTCCGATCT-3′
CGAGAAGGCTAG-5′
3′ -TTACTATGCCGCTGGTGGCTCTAGATGTGAGAAAGGGATGTGCTG′.

13. The kit according to claim 10, characterized in that said high GC clamp linker is formed by annealing two sequences: one sequence is a GC clamp sequence which is 5-50 bp in length; the other sequence contains two parts, one part is reverse complementary to the GC clamp sequence and the other part is reverse complementary to the sequence at the P7 end of the Y-shaped reverse linker.

14. The kit according to claim 10, characterized in that said GC clamp sequences are as follows:
Sequence 1: 5′ TCGACTGCGTG3′
Sequence 2: 5′ CGTATGCCGTCTTCTGCTTGCACGCAGTC3′,
the 5′ end of sequence 1, the 5′ end and the 3′ end of sequence 2 are end closed.

15. The kit according to claim 10, characterized in that said high GC clamp linker is annealed to form the structure of:
5′-CGCTGCGTG-3′
3′- CTGACGCACGTTCGTCTTCTGCCGTATGC-5′.

16. The kit according to claim 10, characterized in that
(a) said two parts of said linker combination are annealed and connected together by the principle of base complementarity during the connecting step and then connected to said gDNA fragment to form the final library; or
(b) said kit is applicable to a sequencing platform employing patterned flow-through technology; or
(c) said sample is selected from the group consisting of a cell line, peripheral blood, cord blood, amniotic fluid, chorion, placenta, umbilical cord, saliva and pharyngeal swab.

17. (canceled)

18. (canceled)

19. A Y-shaped reverse linker, characterized in that said Y-shaped reverse linker sequence is inversely complementary to a normal Y-shaped linker sequence.

20. The Y-shaped reverse linker according to claim 19, characterized in that said Y-shaped reverse linker has the following sequence:
5′pCAAGCAGAAGACGGCATACGAGATNNNNNNNGTGACTGGAGTTCAGA
CGTGTGCTCTTCCGAT*C*T 3′
5′pGATCGGAAGAGCGTCGTGTAGGGAAAGAGTGTAGATCTCGGTGGTCG
CCGTATCATT3′
where N represents random degenerate base A/T/C/G, * represents thio-modification and p represents phosphorylation modification.

21. The Y-shaped reverse linker according to claim 19, characterized in that said Y-shaped reverse linker is annealed to form the following structure:
5′ -CAAGCAGAAGACGGGCATACGAGATNNNNNNNGTGACTGGAGTTCA
GACGTGTGCTCTTCCGATCT-3′
CGAGAAGGCTAG-5′
3′ -TTACTATGCCGCTGGTGGCTCTAGATGTGAGAAAGGGATGTGCTG′.

22. A high GC clamp linker, characterized in that said high GC clamp linker is formed by annealing two sequences: one sequence is a GC clamp sequence, which is 5-50 bp in length; the other sequence contains two parts, one part is reverse-complementary to the GC clamp sequence and the other part is reverse-complementary to the sequence at the P7 end of the Y-shaped reverse linker.

23. The high GC clamp linker according to claim 22, characterized in that said GC linker sequences are as follows:
Sequence 1: 5′ TCGACTGCGTG3′
Sequence 2: 5′ CGTATGCCGTCTTCTGCTTGCACGCAGTC3′,
the 5′ end of sequence 1, the 5′ end and the 3′ end of sequence 2 are end closed.

24. The high GC clamp linker according to claim 23, characterized in that said high GC clamp linker is annealed to form the structure of:
5′-TCGACTGCGTG-3′
3′- CTGACGCACGTTCGTCTTCTGCCGTATGC-5′.

25. The Y-shaped reverse linker according to claim 19, further comprises a high GC clamp linker, characterized in that said high GC clamp linker is formed by annealing two sequences: one sequence is a GC clamp sequence, which is 5-50 bp in length; the other sequence contains two parts, one part is reverse-complementary to the GC clamp sequence and the other part is reverse-complementary to the sequence at the P7 end of the Y-shaped reverse linker.
